SEQUENCE LISTING (25 sequences)

SEQ ID NO: 1 - DNA, 22 bp, Artificial
  Direct primer designed to amplify, in combination with SEQ ID NO2. cDNA of the rat dus6 gene
gggagagatttgctccattcat

SEQ ID NO: 2 - DNA, 23 bp, Artificial
  Reverse primer designed to amplify, in combination with SEQ ID NO1, cDNA of the rat dusp6 gene
aaaagcaaacctattgcctggat

SEQ ID NO: 3 - DNA, 20 bp, Artificial
  dusp6 gene antisense oligomucleotide ODN2
tcaacgtggccatcccgggc

SEQ ID NO: 4 - DNA, 21 bp, Artificial
  dusp6 gene antisense oligonucleotide ODN3
ccaagtggactccctgcaatc

SEQ ID NO: 5 - DNA, 25 bp, Artificial
  Probe sequence of the U42627_at probe set of Affymetrix, the position of said probe in the mRNA sequence of the dusp6 gene being 1557
ttcagtttctcttgggcagcatcga

SEQ ID NO: 6 - DNA, 25 bp, Artificial
  Probe sequence of the U42627_at probe set of Affymetrix, the position of said probe in the mRNA sequence of the dusp6 gene being 1563
ttctcttgggcagcatcgaccaggc

SEQ ID NO: 7 - DNA, 25 bp, Artificial
  Probe sequence of the U42627_at probe set of Affymetrix, the position of said probe in the mRNA sequence of the dusp6 gene being 1623
gtcaccagctgtctgtattagacaa

SEQ ID NO: 8 - DNA, 25 bp, Artificial
  Probe sequence of the U42627_at probe set of Affymetrix, the position of said probe in the mRNA sequence of the dusp6 gene being 1713
ggacagggtatgctgtctagatcca

SEQ ID NO: 9 - DNA, 25 bp, Artificial
  Probe sequence of the U42627_at probe set of Affymetrix, the position of said probe in the mRNA sequence of the dusp6 gene being 1725
ctgtctagatccaggcaataggttt

SEQ ID NO: 10 - DNA, 25 bp, Artificial
  Probe sequence of the U42627_at probe set of Affymetrix, the position of said probe in the mRNA sequence of the dusp6 gene being 1773
agcagggactggacctccatccaga

SEQ ID NO: 11 - DNA, 25 bp, Artificial
  Probe sequence of the U42627_at probe set of Affymetrix, the position of said probe in the mRNA sequence of the dusp6 gene being 1827
ggagcatgtgttccttagggccaca

SEQ ID NO: 12 - DNA, 25 bp, Artificial
  Probe sequence of the U42627_at probe set of Affymetrix, the position of said probe in the mRNA sequence of the dusp6 gene being 1845
ggccacatatggctgtttcctgttg

SEQ ID NO: 13 - DNA, 25 bp, Artificial
  Probe sequence of the U42627_at probe set of Affymetrix, the position of said probe in the mRNA sequence of the dusp6 gene being 1857
ctgtttcctgttgcatctggaacca

SEQ ID NO: 14 - DNA, 25 bp, Artificial
  Probe sequence of the U42627_at probe set of Affymetrix, the position of said probe in the mRNA sequence of the dusp6 gene being 1863
cctgttgcatctggaaccaactata

SEQ ID NO: 15 - DNA, 25 bp, Artificial
  Probe sequence of the U42627_at probe set of Affymetrix, the position of said probe in the mRNA sequence of the dusp6 gene being 1881
aactatattgtcttcagtgaagact

SEQ ID NO: 16 - DNA, 25 bp, Artificial
  Probe sequence of the U42627_at probe set of Affymetrix, the position of said probe in the mRNA sequence of the dusp6 gene being 1887
attgtcttcagtgaagactgattca

SEQ ID NO: 17 - DNA, 25 bp, Artificial
  Probe sequence of the U42627_at probe set of Affymetrix, the position of said probe in the mRNA sequence of the dusp6 gene being 1935
gagattttagctctgtatttgtggt

SEQ ID NO: 18 - DNA, 25 bp, Artificial
  Probe sequence of the U42627_at probe set of Affymetrix, the position of said probe in the mRNA sequence of the dusp6 gene being 1941
ttagctctgtatttgtggtatcggt

SEQ ID NO: 19 - DNA, 25 bp, Artificial
  Probe sequence of the U42627_at probe set of Affymetrix, the position of said probe in the mRNA sequence of the dusp6 gene being 2007
aatatttgatcttcacttgagagtg

SEQ ID NO: 20 - DNA, 25 bp, Artificial
  Probe sequence of the U42627_at probe set of Affymetrix, the position of said probe in the mRNA sequence of the dusp6 gene being 2013
tgatcttcacttgagagtgtttgtt

SEQ ID NO: 21 - DNA, 20 bp, Artificial
  Direct primer designed to amplify, in combination with SEQ ID NO 22, cDNA of the rat glyceraldehyde 3-phosphate dehydrogenase gene
aaggctggggctcacctgaa

SEQ ID NO: 22 - DNA, 21 bp, Artificial
  Reverse primer designed to amplify, in combination with SEQ ID NO 21, cDNA of the rat glyceraldehyde 3-phosphate dehydrogenase gene
ggcatggactgtggtcatgag

SEQ ID NO: 23 - DNA, 20 bp, Artificial
  A dusp6 gene antisense oligonucleotide
cgttgagccacgccaccgtc

SEQ ID NO: 24 - DNA, 23 bp, Artificial
  Primer used in quantitative real-time RT-PCR for evaluating the expression of dusp6 gene in post-mortem human tissue samples from optic nerve
cctgaggccatttctttcataga

SEQ ID NO: 25 - DNA, 23 bp, Artificial
  Primer used in quantitative real-time RT-PCR for evaluating the expression of dusp6 gene in post-mortem human tissue samples from optic nerve
gtcacagtgactgagcggctaat

(19) United States
(12) Patent Application Publication
(10) Pub. No.: US 2008/0113347 A1
(43) Pub. Date: May 15, 2008

(54) Method for the in Vitro Diagnosis and Prognosis of Demyelinating Diseases, and for the Development of Drugs Against Demyelinating Diseases

(76) Inventors: Ainara Vallejo Illarramendi, Vizcaya (ES); Antonio Martinez Martinez, Vizcaya (ES); Usue Ariz Lopez de Castro, Vizcaya (ES); Lourdes Osaba Ortiz de Mendibil, Vizcaya (ES); Corina Junquera Sanchez-Vallejo, Vizcaya (ES); Simon Santa Cruz, West Sussex (GB); Pedro Escudero Garcia de Galdeano, Vizcaya (ES); Jorge Ochoa Garay, Vizcaya (ES); Laureano Simon Buela, Gipuzkoa (ES); Carlos Matute Almau, Vizcaya (ES); Elena Alberdi Alfonso, Vizcaya (ES); Maria Victoria Sanchez Gomez, Vizcaya (ES); Gaskon Ibarretxe Bilbao, Vizcaya (ES); Estibaliz Etxebarria Galnares, Alava (ES)

Correspondence Address: MOORE & VAN ALLEN PLLC, P.O. BOX 13706, Research Triangle Park, NC, 27709, US

(21) Appl. No.: 10/596,857

(22) PCT Filed: Dec. 28, 2004
(86) PCT No.: PCT/EP04/14814

§ 371 Date: Jan. 31, 2007

(30) Foreign Application Priority Data
Dec. 30, 2003 (EP) 03380310.7

(51) Int. Cl. C12Q 1/68; C07H 21/04

(52) U.S. Cl. 435/6; 536/22.1

(57) ABSTRACT

The present invention refers to an in vitro method for detecting the presence of demyelinating diseases in an individual, for determining the stage or severity of said diseases in the individual, or for monitoring the effect of the therapy administered to an individual suffering said diseases; to the search, identification, development and evaluation of efficacy of compounds for therapy of said diseases for the purpose of developing new drugs; as well as to agents inhibiting DUSP6 protein expression and/or activity, and/or the effects of this expression. The methods and agents of the invention are preferably applied to multiple sclerosis.

### FIELD OF THE INVENTION

The present invention refers to an in vitro method for detecting the presence of demyelinating diseases in an individual, for detecting the stage or severity of said diseases in the individual, or for monitoring the effect of the therapy administered to an individual presenting said diseases; to the search, identification, development and evaluation of efficacy of compounds for therapy of said diseases for the purpose of developing new medicaments; as well as to agents inhibiting the expression and/or activity of the DUSP6 protein, and/or to the effects of this expression. The methods and agents of the invention are preferably applied to multiple sclerosis.

### BACKGROUND OF THE INVENTION

Demyelinating diseases are those in which the main pathogenic process causes the destruction of the myelin sheath, which is necessary for the integrity of central nervous system cells. The main demyelinating diseases are: multiple sclerosis, Devic's syndrome, Baló disease, Marchiafava-Bignami disease, central pontine myelinolysis, acute disseminated encephalomyelitis, acute necrotizing hemorrhagic encephalomyelitis. Among them, multiple sclerosis is the most frequent disease due to alteration of the myelin in the central nervous system and, with the exception of trauma, it is the most frequent cause of neurological impairment in young adults. It is estimated that approximately 75% of the cases of demyelinating diseases correspond to multiple sclerosis.

Multiple sclerosis (MS) is the most frequent demyelinating disease of the central nervous system. It affects 1.5 million people worldwide, and its symptoms generally occur in young adults, therefore its consequences at a personal and socioeconomic level are very severe (Noseworthy et al. 2000 New Engl. J. Med., 343, 938-952).

Susceptibility to MS is due to unknown genetic and environmental factors. Prevalence of the disease is about 50 to 100 persons every 100,000 habitants in high risk regions, which are mainly located in northern areas of the northern hemisphere in Europe and America. The risk of suffering MS increases 10-20 fold in first degree relatives of patients, and concordance between monozygotic (genetically identical) twins increases up to 30-35%, whereas in dizygotic twins it only reaches 2-5% (Noseworthy et al. 2000 New Engl. J. Med., 343, 938-952). Genetic susceptibility is not characterized. To date, there is evidence that it can reside in some polymorphism of the genes encoding human leukocyte antigens (HLA), myelin oligodendrocyte glycoprotein (MOG) and other genes of chromosomes 10 and 15 (Steinman 2001, Nature Immunol 2, 762-764).

There is a consensus among MS researchers according to which the disease has two stages, an initial inflammatory phase of an autoimmune nature, and another secondary progressive neurodegenerative phase. In the first phase, activated T cells cross the hematoencephalic barrier, and once inside the central nervous system, they release proinflammatory cytokines triggering an immunological cascade ending in the destruction of the myelin and death of the oligodendrocytes. Knowledge of the autoimmune process with certain detail has served to develop agents of an immunomodulating nature, therapeutic efficacy of which is very modest. Until now, different targets for intervention during the inflammatory phase of MS (Zamvil and Steinman, 2003, Neuron 38, 685-688) have been disclosed. Among them are those which are focused on reducing inflammation of the nervous system initiated by the activation of the myelin-specific T cells, promoting autoimmunity particularly against components of the myelin, entering the central nervous tissue and releasing in it proinflammatory cytokines such as interferon-γ and tumor-α necrosis factor. The immunomodulator interferon-1β, approved for the treatment of remitting-recurrent MS, also prevents cellular interactions leading to the penetration of activated T cells through the vascular endothelium. Other treatments in clinical trial phase are focused on neutralizing the activity of proinflammatory cytokines and/or to enhance anti-inflammatory ones. However, no medication has been generated which delays or stops the progression of the neurodegenerative phase of the disease which takes a course with progressive neurological degeneration, and which is characterized by the occurrence of severe demyelinating lesions in the white substance with massive oligodendrocyte loss, atrophy and severe axonal damage.

Glutamic acid is the main central nervous system exciter neurotransmitter. It activates a broad family of well characterized receptors at a molecular and functional level (Dingledine et al., 1999, Pharmacol. Rev. 51:7-61.), and it is removed form the extracellular space by means of carriers (Danbolt, 2001, Prog. Neurobiol 65, 1-105). Decrease of the efficacy of glutamic acid transport may cause the receptor overexcitation and trigger neuronal death. This phenomenon is relevant in the etiology of some neurological disorders, among which neurodegenerative diseases are included (Lee et al., 1999, Nature 399:A7-14).

Oligodendrocytes are the cells responsible for central nervous system myelinization, and their death causes severe alterations in nervous communication. Just like neurons, oligodendrocytes express glutamic acid receptors and transporters thereof, the molecular entity of which has been characterized in recent years. Likewise, it has been observed that overactivation of glutamatergic receptors causes oligodendroglial death. These results have led to postulate that glutamic acid homeostasis is fundamental for preventing possible oligodendroglial excitotoxicity, and that the latter may be a component in the etiopathogeny of demyelinating diseases (revised in Matute et al., 2001, Trends Neurosci. 24:224-230; Matute et al., 2002, Eur. J. Pharmacol, 447:239-246). Therefore, full understanding of the excitotoxic process may provide important data which, among other things, serve to establish possible therapeutic targets to act on by means of the development of new drugs.

Excitotoxicity can be mediated by glutamatergic receptors of the NMDA, AMPA or kainate type. Only the last two participate in oligodendrocytes, as these cells lack NMDA receptors (Sánchez-Gómez and Matute, 1999, Neurobiol. Dis. 6:475-485), and excitotoxic death of these cells is caused by an overload in cytosolic calcium levels (Alberdi et al., 2002, Neurobiol. Dis. 9:234-243). Excessive increase of this metabolite causes the activation of proteases, lipases and endonucleases, which can damage structural proteins, the membrane and DNA, respectively. In turn, excitotoxic cell death can occur by means of apoptosis or necrosis. However, the cellular mechanisms leading to either process, and the molecules intervening, are not well known.

The existence of in vivo experimental excitotoxic damage models allow examining the relevance of the findings obtained with the studies carried out in oligodendrocyte cultures. Thus, the infusion of excitotoxins in the optic nerve causes demyelination in plaques similar to MS lesions (Matute, 1998, Proc. Natl. Acad. Sci. USA 95:10229-10234). On the other hand, ischemic processes affecting the central nervous system cause massive excitotoxic damage to the oligodendroglial population (Li and Stys, 2000, J. Neurosci. 20:1190-1198). Finally, experimental autoimmune encephalomyelitis causes demyelination which can be largely reduced by means of the administration of glutamatergic receptor antagonists (Pitt et al., 2000, Nat. Med. 6:67-70; Smith et al., 2000, Nat. Med. 6:62-66). As a whole, experimental studies of demyelinating diseases in cellular and animal models indicate that excitotoxic death of oligodendrocytes is one component in the etiology of these diseases, and particularly of MS.

The DUSP (abbreviation for dual specificity protein tyrosine phosphatases) phosphatase family is involved in the regulation of kinases activated by extracellular signals (ERK), by cellular stress (JNK/SAPK) and by mitogens (MAPK). These kinases are activated by reversible phosphorylation of threonines and tyrosines and are inactivated by DUSPs which dephosphorylate these same moieties (Muda et al., 1996, J. Biol. Chem. 271:27205-27208). Just like the kinases that they regulate, DUSPs have been related with cell differentiation, regeneration and apoptosis. In relation to this last biological process, it has been observed that DUSP6 (also known as MKP-3, or MAP kinase phosphatase-3), (GeneBank code rat NM_{—}053883, human NM_{—}001946) has a cytosolic localization and induces apoptosis in endothelial cells in response to proinflammatory cytokines due to a mechanism which, in the last extreme, causes proteolysis of the anti-apoptotic Bcl-2 protein (Rossig et al., 2000, J. Biol. Chem. 275:25502-25507). However, the dusp6 gene, or the protein it encodes, DUSP6, has never been associated to apoptosis processes in nervous cells, or to demyelinating processes or diseases.

The authors of the present invention have discovered, after laborious research and using different techniques (DNA-chips and quantitative RT-PCR to measure gene expression levels, as well as inhibition assays of cell death due to apoptosis) that the expression of the dusp6 gene increases after excitotoxic stimuli in a pure oligodendrocyte culture, and that the blocking of its expression rescues these cells from death by apoptosis; and that the dusp6 gene expression is increased in post-mortem brain samples from human individuals affected with multiple sclerosis when compared to the expression in post-mortem brain samples from human individuals not affected with multiple sclerosis. These evidences convert dusp6 and the protein it encodes, DUSP6, into a useful target for the development of new in vitro methods of search, identification, development and evaluation of efficacy of compounds for therapy of demyelinating diseases, particularly for multiple sclerosis, for the purpose of developing new drugs.

Therefore, the present invention provides a high sensitivity in vitro method for detecting the presence of demyelinating diseases in an individual, for determining the stage or severity of said diseases in the individual, for monitoring the effect of the therapy administered to an individual presenting said diseases, or for deciding which patients suffering from demyelinating diseases are susceptible to being treated with drugs developed for inhibiting the expression and/or activity of the DUSP6 protein, and/or the effects of this expression The methods and agents of the invention are preferably applied to multiple sclerosis.

### OBJECT OF THE INVENTION

The main object of the present invention is the development of an in vitro method for detecting the presence of demyelinating diseases, for determining the stage or severity of said diseases in the individual, or for monitoring the effect of the therapy administered to an individual presenting said diseases.

A second object of the present invention is an in vitro method for searching for, identifying, developing and evaluating efficacy of compounds for the therapy of demyelinating diseases.

An additional object of the invention is based on the use of sequences derived from the dusp6 gene for the in vitro diagnosis and prognosis of demyelinating diseases; as well as for the search, identification, development and evaluation of efficacy of compounds for the therapy of said diseases, particularly, for the therapy of the neurodegenerative phase of said diseases.

Another object of the present invention consists of providing agents characterized in that they inhibit the expression and/or activity of the DUSP6 protein for the treatment of demyelinating diseases, particularly, for the treatment of the neurodegenerative phase of said diseases.

Another object of the invention relates to the use of said agents that inhibit DUSP6 protein expression and/or activity, or that inhibits the lethal effects of induction of DUSP6 protein expression, in the manufacturing of a pharmaceutical composition for the treatment demyelinating diseases, particularly, for the treatment of the neurodegenerative phase of said demyelinating diseases.

Also object of the invention is a pharmaceutical composition comprising one or several therapeutic agents together with a pharmaceutically acceptable excipient for the treatment of demyelinating diseases, particularly, for the treatment of the neurodegenerative phase of said demyelinating diseases.

Lastly, another object of the invention is a kit for carrying out the present invention.

### DESCRIPTION OF THE FIGURES

FIG. 1 shows a melting curve of the PCR product of reference gene, GADPH (Tm=85° C.) (FIG. 1a) and of the target gene, dusp6 (Tm=77° C.) (FIG. 1b), in measurement experiments of gene expression by real time quantitative RT-PCR. Temperature (° C.) is represented on the axis of abscissas and fluorescence (d(F1)/dT) is represented on the axis of ordinates.

FIG. 2 shows the calculation of the amplification efficacy of the dusp6 PCR reactions in gene expression measurement experiments by real time quantitative RT-PCR. The standard line obtained for dusp6 data. The concentration logarithm is shown on the axis of abscissas and the crossing point is shown on the axis of ordinates.

FIG. 3 shows the effect of antisense oligonucleotides against caspase-3 (FIG. 3a) and against dusp6 (FIG. 3b) on the oligodendroglial death process after stimulation with AMPA 10 μM+CTZ 100 μM for 15 min. ** p<0.01, *p<0.05, n=3-5.

FIG. 4 shows that inhibition of the MAPK/ERK kinase pathway potentiates oligodendrocyte death induced by AMPA receptor stimulation; in fact, whereas the MEK inhibitor UO126 did not affect oligodendroglial viability under control conditions, oligodendroglial cell death induced by AMPA 10 μM+CTZ 100 μM was increased three-fold in the presence of UO126 (p<0.05; n=3 in triplicate).

FIG. 5 shows the alignment of human (NM_{—}001946) and rat (NM_{—}053883) dusp6 nucleotide sequences.

FIG. 6 shows the alignment of human (NP_{—}001937) and rat (NP_{—}446335) DUSP6 amino acid sequences.

FIG. 7 shows dusp6 gene expression levels in postmortem human optic nerve samples. MS means multiple sclerosis.

### DETAILED DESCRIPTION OF THE INVENTION

To facilitate comprehension of the present patent application, the meaning of some terms and expressions in the context of the invention will be explained below:

The terms “subject” or “individual” refer to members of mammal species, and includes, but is not limited to, domestic animals, primates and humans; the subject is preferably a human being, male or female, of any age or race.

The expression “demyelinating diseases” refers to those diseases in which the main pathogenic process causes the destruction of the myelin, which is the lipoprotein layer covering nerves and facilitates the transmission of impulses through nervous fibers. In the scope of the present invention, demyelinating diseases include: multiple sclerosis, Devic's syndrome, Baló disease, Marchiafava-Bignami disease, central pontine myelinolysis, acute disseminated encephalomyelitis, acute necrotizing hemorrhagic encephalomyelitis.

The term “gene” refers to a molecular chain of deoxyribonucleotides encoding a protein.

The term “DNA” refers to deoxyribonucleic acid. A DNA sequence is a deoxyribonucleotide sequence.

The term “cDNA” refers to a nucleotide sequence complementary to an mRNA sequence.

The term “RNA” refers to ribonucleic acid. An RNA sequence is a ribonucleotide sequence.

The term “mRNA” refers to messenger ribonucleic acid, which is the fraction of total RNA translated into proteins.

The phrase “mRNA transcribed from” refers to transcription of the gene (DNA) into mRNA as a first step for the gene to be expressed and translated into a protein.

The term “nucleotide sequence” indistinctively refers to a ribonucleotide (RNA) or deoxyribonucleotide (DNA) sequence.

The term “protein” refers to a molecular chain of amino acids bonded by covalent or non-covalent bonds. The term includes all the post-translation modification forms, for example, glycosylation, phosphorylation or acetylation.

The terms “peptide” and “polypeptide” refer to molecular chains of amino acids representing a protein fragment. The terms “protein” and “peptide” are used indistinctively.

The term “antibody” refers to a glycoprotein exhibiting a specific binding activity for a particular protein, which is called “antigen”. The term “antibody” comprises monoclonal antibodies, or polyclonal antibodies, intact or fragments of them; and it includes human, humanized and non-human origin antibodies. “Monoclonal antibodies” are homogenous populations of highly specific antibodies directed against a single site or antigen “determinant”. “Polyclonal antibodies” include heterogeneous populations of antibodies directed against different antigen determinants.

The term “epitope”, as it is used in the present invention, refers to an antigen determinant of a protein, which is the amino acid sequence of the protein which a specific antibody recognizes.

The term “solid phase”, as it is used in the present invention, refers to a non-aqueous matrix which the antibody can bind to. Examples of solid phase materials include glass, polysaccharides, for example agarose, polyacrylamide, polystyrene, polyvinyl alcohol and silicones. Examples of solid phase forms are an assay plate well or purification column.

The term “oligonucleotide primer”, as it is used in the present invention, refers to a nucleotide sequence, which is complementary of a dusp6 gene nucleotide sequence. Each primer hybridizes with its target nucleotide sequence and acts as a DNA polymerization start site.

The term “probe”, as it is used in the present invention, refers to a complementary nucleotide sequence of a dusp6 gene nucleotide sequence, which can be used to detect that nucleotide sequence derived from the dusp6 gene.

The term “therapeutic target” refers to nucleotide or peptide sequences against which a drug or therapeutic compound can be designed and clinically applied.

The term “antagonist” refers to any molecule inhibiting the biological activity of the antagonized molecule. Examples of antagonist molecules include, among others, proteins, peptides, natural peptide sequence variations and small organic molecules (having a molecular weight of less than 500 Daltons).

The present invention is based on the discovery that both dusp6 gene expression and the DUSP6 protein concentration are increased during cell death of oligodendrocytes, which are the cells responsible for central nervous system myelinization; on the discovery that blocking dusp6 gene expression with a antisense RNA specific of this gene blocks the death process of oligodendrocytes, therefore blocking the demyelinization process; and on the discovery that dusp6 gene expression is increased in post-mortem brain samples from human individuals affected with multiple sclerosis when comparing the expression in post-mortem brain samples form human individuals not affected with multiple sclerosis.

In this sense, the present invention, in the first place, provides an in vitro method for detecting the presence of demyelinating diseases in an individual, for determining the stage or severity of said diseases in the individual, or for monitoring the effect of the therapy administered to an individual presenting said diseases, comprising:

a) detection and/or quantification of the DUSP6 protein, of the dusp6 gene mRNA or the corresponding cDNA in a sample from said individual, and

b) comparison of the DUSP6 protein amount, of the dusp6 gene mRNA amount, or of the corresponding cDNA amount detected in a sample from an individual, with the DUSP6 protein amount, with dusp6 gene mRNA amount, or with the corresponding cDNA amount detected in the samples of control individuals, or in previous samples from the same individual, or, with normal reference values.

The method provided by the present invention is of a high sensitivity and specificity, and is based on the fact that subjects or individuals diagnosed with a demyelinating disease, especially multiple sclerosis, present high levels of mRNA transcribed from the dusp6 gene (high dusp6 gene expression levels), or high concentrations of the protein encoded by the dusp6 gene (DUSP6 protein), in comparison with corresponding levels in samples from subjects with no clinical history of demyelinating diseases.

The present method comprises a step for obtaining the sample from the individual. It is possible to work with different fluid samples such as, for example: serum, urine, saliva, feces or cerebrospinal fluid. The sample can be obtained by any conventional method, preferably by surgical resection.

The samples can be obtained from subjects previously diagnosed, or not diagnosed, with a demyelinating disease, preferably multiple sclerosis; or also from a subject undergoing treatment, or who has been previously treated for a demyelinating disease, particularly for multiple sclerosis.

The present method also comprises a sample extraction step, either for obtaining the protein extract from the latter, or for obtaining the total RNA extract. One of these two extracts represents the working material for the following phase. Total protein or total RNA extraction protocols are well known by a person skilled in the art (Chomczynski P. et al., Anal. Biochem., 1987, 162: 156; Chomczynski P., Biotechniques, 1993, 15: 532).

Any conventional test can be used within the framework of the invention for detecting a demyelinating disease, as long as it measures in vitro levels of mRNA transcribed from the dusp6 gene or its complementary cDNA, or the DUSP6 protein concentration, in samples taken from the individuals to be analyzed and from control individuals.

Therefore, this invention provides a method for detecting the presence of demyelinating diseases, especially multiple sclerosis, in an individual, for determining the stage or severity of said diseases, or for monitoring the effect of the therapy administered to an individual presenting said diseases, either based on the DUSP6 protein concentration, or on the measurement of the dusp6 gene expression level.

In the event that the DUSP6 protein is to be detected, the method of the invention comprises a first step of contacting the protein extract of the sample with a composition of one or more specific antibodies against one or more epitopes of the DUSP6 protein, and a second step for quantifying the complexes formed by antibodies and the DUSP6 protein.

There is a wide variety of immunological assays available for detecting and quantifying the formation of specific antigen-antibody complexes; numerous competitive and non-competitive protein binding assays have previously been disclosed, and a large number of these assays are commercially available.

Thus, the DUSP6 protein can be quantified with antibodies such as, for example: monoclonal antibodies, polyclonal antibodies, intact or recombinant fragments thereof, “combibodies” and Fab or scFv antibody fragments, specific against the DUSP6 protein; these antibodies being human, humanized or of non-human origin. The antibodies used in these assays can be labeled or not; the unlabeled antibodies can be used in agglutination assays; the labeled antibodies can be used in a wide variety of assays. Marker molecules which can be used to label antibodies include radionucleotides, enzymes, fluorophores, chemoluminescent reagents, enzyme substrates or cofactors, enzyme inhibitors, particles, colorants and derivatives.

There is a wide range of well known assays which can be used in the present invention using unlabeled antibodies (primary antibody) and labeled antibodies (secondary antibody); included among these techniques are the Western-blot or Western transfer, ELISA (Enzyme-Linked Immunosorbent Assay), RIA (Radioimmunoassay), competitive EIA (Competitive Enzyme Immunoassay), DAS-ELISA (Double Antibody Sandwich-ELISA), immunocytochemical or immunohistochemical techniques, techniques based on the use of biochips or microarrays of proteins including specific antibodies, or assays based on colloidal precipitation in formats such as dipsticks. Other ways to detect and quantify the DUSP6 protein include affinity chromatography techniques, ligand binding assays or lectin binding assays.

The immunoassay preferred in the method of the invention is a double antibody sandwich-ELISA (DAS-ELISA) assay. In this immunoassay, any antibody or combination of antibodies specific against one or more epitopes of the DUSP6 protein can be used. As an example of one of the many possible formats of this assay, a monoclonal or polyclonal antibody, or a fragment of this antibody, or a combination of antibodies coating a solid phase, are put into contact with the sample to be analyzed, and incubated for a certain time and under conditions suitable for forming antigen-antibody complexes. After a rinsing under conditions suitable for removing the non-specific complexes, an indicator reagent, comprising a monoclonal or polyclonal antibody, or a fragment of this antibody, or a combination of these antibodies, bonded to a signal-generating compound, is incubated with the antigen-antibody complexes under suitable conditions and time. The presence of the DUSP6 protein in the sample to analyze is detected and quantified, if any, by measuring the generated signal. The DUSP6 protein amount present in the sample to analyze is proportional to that signal.

In the event that the mRNA or cDNA corresponding to the dusp6 gene, and not the protein, is to be detected, the method of the invention for detecting in vitro a demyelinating disease has different steps. Thus, once the sample is obtained and the total RNA is extracted, according to the method of the invention, detection of the mRNA or corresponding cDNA of the dusp6 gene is carried out, comprising a first amplification step of the total RNA extract or corresponding cDNA synthesized by mRNA reverse transcription, and a second quantification step of the amplification product of the mRNA or cDNA of the dusp6 gene.

One example of mRNA amplification consists of reverse transcription mRNA into cDNA (RT), followed by Polymerase Chain Reaction (PCR) using oligonucleotide primers, the sequences of the primers used being SEQ ID NO:1 and SEQ ID NO:2; PCR is a technique for amplification of a certain nucleotide sequence (target) contained in a nucleotide sequence mixture. In PCR, an oligonucleotide primer pair excess is used, which primers hybridize with the target nucleotide sequence complementary strands. Then, an enzyme with polymerase activity (DNA Taq Polimerase) extends each primer, using the target nucleotide sequence as a template. The extension products are then converted into target sequences after the disassociation of the original target strand. New primer molecules hybridize and the polymerase extends them; the cycle is repeated to exponentially increase the number of target sequences. This technique is disclosed in U.S. Pat. No. 4,683,195 and U.S. Pat. No. 4,683,202. Different methods have previously been disclosed for detecting and quantifying PCR amplification products, any of which can be used in this invention. In a preferred method of the invention, the amplified product is detected by agarose gel electrophoresis in the following manner: five microliters of the amplification product are subjected to separation by agarose gel electrophoresis at a 2% concentration in a TAE 1× buffer at 100 Volts for one hour. After electrophoresis, the gel is stained with ethidium bromide, and the amplification product is visualized when illuminating the gel with ultraviolet (uv) light; as an alternative to staining, and a preferred embodiment, the amplified product can be transferred to a nylon membrane by Southern blotting or Southern transfer techniques to be detected with a suitably marked, specific probe of the dusp6 gene cDNA.

In another example, mRNA detection is carried out by transferring the mRNA to a nylon membrane by means of transfer techniques such as, for example, Northern-blot or Northern transfer, and detecting it with specific probes of the mRNA or the corresponding cDNA of the dusp6 gene.

In a particular embodiment, amplification and quantification of the mRNA corresponding to the dusp6 gene is carried out at the same time by means of real time quantitative RT-PCR (Q-PCR).

The final step of the method of the invention for detecting in vitro a demyelinating disease in a sample from an individual comprises comparing the DUSP6 protein amount, the dusp6 gene mRNA amount, or the corresponding cDNA amount detected in a sample from an individual, with the DUSP6 protein amount, the dusp6 gene mRNA amount, or the corresponding cDNA amount detected in control subject samples or with normal reference values.

As its second object, the invention provides an in vitro method for identifying and evaluating the efficacy of agents for therapy of demyelinating diseases, especially multiple sclerosis:
a) treating rat optic nerve oligodendrocyte primary culture with stimuli relevant to demyelinating diseases, preferably with excitotoxic stimuli such as Ampa or Kainate,
b) detecting and quantifying changes in the dusp6 gene or DUSP6 protein expression in cells of the culture in response to said stimuli,
c) putting the stimulated primary oligodendrocyte culture obtained in step a) into contact with the candidate compound under the conditions and for the time suitable for permitting them to interact,
d) detecting and quantifying the dusp6 gene or DUSP6 protein expression levels, and
e) comparing the expression levels obtained in step d) with the corresponding levels in pure stimulated oligodendrocyte cultures not treated with the candidate compound.

Quantification of the dusp6 gene or DUSP6 protein expression levels is carried out in a manner similar to how it is indicated in the method of the invention for detecting in vitro the presence of a demyelinating disease in an individual.

When an agent decreases dusp6 gene expression levels or reverts the effects of the increased expression of said gene, preferably blocking the cell death process, this agent is converted into a candidate for the therapy of demyelinating diseases, especially multiple sclerosis, particularly a candidate for the therapy of the neurodegenerative phase of demyelinating diseases, preferably, multiple sclerosis.

Another aspect of the invention refers to the use of nucleotide or peptide sequences derived from the dusp6 gene in methods of search, identification, development and evaluation of efficacy of compounds for therapy of the neurodegenerative phase of demyelinating diseases, especially multiple sclerosis. Within the search methods, the importance acquired recently by drug screening methods, based on the binding, competitive or not, of the potential drug molecule to the therapeutic target, stands out.

Another additional object of the invention refers to the use of nucleotide or peptide sequences derived from the dusp6 gene for detecting the presence of demyelinating diseases, especially multiple sclerosis, for determining the stage or severity of said diseases in the individual, or for monitoring the effect of the therapy administered to an individual presenting said diseases.

Another object of the invention consists of providing agents characterized in that they inhibit DUSP6 protein expression and/or activity. Said agents are potentially useful in therapy of demyelinating diseases, particularly they are potentially useful in therapy of the neurodegenerative phase of said demyelinating diseases preferably, multiple sclerosis. These agents, which can be identified and evaluated according to the present invention, can be chosen from the group formed by:

a) an antibody, or combination of antibodies, specific against one or more epitopes present in the DUSP6 protein, preferably a human or humanized monoclonal antibody; also being possible a fragment of the antibody, a simple-chain antibody or an anti-idiotype antibody,

b) cytotoxic agents, such as toxins, molecules with radioactive atoms, or chemotherapeutic agents, which include, without limitation, small organic and inorganic molecules, peptides, phosphopeptides, antisense molecules, ribozymes, siRNAs, triple helix molecules, etc., inhibiting DUSP6 protein expression and/or activity, such as, for example, the dusp6 specific antisense oligonucleotides SEQ ID NO:3 and SEQ ID NO:4, which inhibit cell death of oligodendrocytes treated with AMPA, or any antisense oligonucleotide with an homology with said molecule exceeding 50%, or any dusp6 specific antisense oligonucleotide inhibiting its expression, and

c) DUSP6 protein antagonist compounds inhibiting one or more of the DUSP6 protein functions.

The dusp6 specific antisense oligonucleotides of SEQ ID NO:3 and SEQ ID NO:4, which inhibit cell death of oligodendrocytes treated with AMPA (Example 3), constitute an additional object of the instant invention. Said products are useful for treating demyelinating diseases, in particular the neurodegenerative phase of a demyelinating diseases, such as multiple sclerosis.

Another object of the invention relates to the use of the above mentioned agent that inhibits DUSP6 protein expression and/or activity, or that inhibits the lethal effects of induction of DUSP6 protein expression, in the manufacturing of a pharmaceutical composition for the treatment of the neurodegenerative phase of demyelinating diseases, especially multiple sclerosis. In a particular embodiment, said pharmaceutical composition further contains another active ingredient, preferably one which inhibits DUSP6 protein function.

Lastly, a pharmaceutical composition comprising a therapeutically effective amount of one or several agents of those previously mentioned, together with one or more excipients and/or carrier substances also constitutes an object of the present invention. Furthermore, said composition may contain any other active ingredient inhibiting DUSP6 protein function.

The excipients, carrier substances and auxiliary substances must be pharmaceutically and pharmacologically tolerable, such that they can be combined with other components of the formulation or preparation and do not exercise adverse effects on the treated organism. Pharmaceutical compositions or formulations include those which are suitable for oral or parenteral administration (including subcutaneous, intradermal, intramuscular and intravenous), although the best administration route depends on the patient's condition. The formulations can be in single dose form. The formulations are prepared according to methods known in the field of pharmacology. The amounts of active substances to be administered can vary according to the particularities of therapy.

Another object of the invention consists in a kit for carrying out the present invention. Thus, an embodiment of the present invention provides a kit that comprises an antibody specific against the DUSP6 protein and a carrier in suitable packing, wherein said antibody is, for example, a monoclonal antibody, a polyclonal antibody, an intact or recombinant fragment thereof, a “combibody” or a Fab or scFv antibody fragment; said antibody being human, humanized or of non-human origin. Said antibody can be labeled or not; the unlabeled antibody can be used in agglutination assays; the labeled antibody can be used in a wide variety of assays. Marker molecules which can be used to label antibodies include radionucleotides, enzymes, fluorophores, chemoluminescent reagents, enzyme substrates or cofactors, enzyme inhibitors, particles, colorants and derivatives. The kit can also contain a combination of said antibodies.

In another embodiment the kit of the invention comprises a primer pair designed to specifically amplify a nucleic acid having a sequence that is specific to the dusp6 gene. The sequence of the primer pair can be determined from the sequence of the corresponding duspe6 gene by employing bioinformatic tools. The sequence of said primer pair is preferably selected from SEQ ID NO:1 and SEQ ID NO:2.

The kits of the invention can be employed to detect the presence of demyelinating diseases in an individual, to determine the stage or severity of said conditions in an individual or to monitor the effect of the therapy administered to the individual with said conditions.

The following examples illustrate the invention.

### EXAMPLES

### Example 1

### Differential Analysis of dusp6 Gene Expression in Oligodendrocyte Samples Using the Microarrays Rat Genome U34 DNA Arrays

### 1.1. Materials and Methods

Microarrays. The microarrays GeneChip Test 3 (Affymetrix, Santa Clara) were used which allow testing the quality of the RNA prior to the expression analysis with GeneChip Rat Genome U34 set (Affymetrix, Santa Clara), representing 3322 complete gene sequences. The dusp6 gene is represented in the microarray by probe set U42627_at of Affymetrix, which are sense oligonucleotides of 25 nucleotides in length designed on the basis of GeneBank U42627 sequence (Table 1).

TABLE 1
Description of probes corresponding to U42627_at probe set.

| Consecutive | | Position of probe |
|---|---|---|
| order of | | in mRNA sequence |
| probes | Probe sequence (5′-3′) | of gene |
| 1 | SEQ ID NO: 5 | 1557 |
| 2 | SEQ ID NO: 6 | 1563 |
| 3 | SEQ ID NO: 7 | 1623 |
| 4 | SEQ ID NO: 8 | 1713 |
| 5 | SEQ ID NO: 9 | 1725 |
| 6 | SEQ ID NO: 10 | 1773 |
| 7 | SEQ ID NO: 11 | 1827 |
| 8 | SEQ ID NO: 12 | 1845 |
| 9 | SEQ ID NO: 13 | 1857 |
| 10 | SEQ ID NO: 14 | 1863 |
| 11 | SEQ ID NO: 15 | 1881 |
| 12 | SEQ ID NO: 16 | 1887 |
| 13 | SEQ ID NO: 17 | 1935 |
| 14 | SEQ ID NO: 18 | 1941 |
| 15 | SEQ ID NO: 19 | 2007 |
| 16 | SEQ ID NO: 20 | 2013 |

Samples. The samples studied corresponded to primary oligodendrocyte cultures obtained from the optic nerve of 12-day old rats. To prevent inter-individual variations, each culture corresponded to a 10-animal cell pool. Treatments were made in duplicate with 10 μM AMPA (α-amino-3-hydroxy-5-methyl-4-isoxazolepropionic acid) or 3 μM kainate. The duration of treatments was 2, 10 and 15 minutes, the cells being collected immediately after treatment in the first two cases and 1 hour after the 15-minute treatment. Treatment for 10 and 15 minutes with AMPA and with kainate causes apoptosis, whereas 2-minute treatment was considered sublethal due to scarce treatment duration. After treatments, the cells were collected in TRIzol® Reagent (Life Technologies) and were stored at −80° C. until total RNA extraction.

GeneChip Gene Expression Analysis

The analysis was carried out with total RNA from oligodendrocyte cultures. Each sample corresponded to the mixture of total RNAs from two cultures and were called the following:

control: untreated cultured oligodendrocytes

ampa 2: oligodendrocytes treated for 2 minutes with 10 μM AMPA and collected after treatment

ampa 10: oligodendrocytes treated for 10 minutes with 10 μM AMPA collected after treatment

ampa 15: oligodendrocytes treated for 15 minutes with 10 μM AMPA and collected 60 minutes later

Kainate 2: oligodendrocytes treated for 2 minutes with 3 μM kainate and collected after treatment

Kainate 10: oligodendrocytes treated for 10 minutes with 3 μM kainate and collected after treatment

Kainate 15: oligodendrocytes treated for 15 minutes with 3 μM kainate and collected 60 minutes later

cRNA Synthesis

The total RNA of each one of the cultures was obtained by homogenizing the cells in TRIzol® Reagent (Life Technologies) following the supplier's recommendations. The resulting total RNA was cleaned with the RNeasy Mini kit (QIAGEN) (Chomczynski P. et al., Anal. Biochem., 1987, 162: 156; Chomczynski P., Biotechniques, 1993, 15: 532). 2 μg from each total RNA preparation were used as starting material for the synthesis of biotinylated cRNA with the MessageAmp™ aRNA kit (Ambion).

Array Hybridization and Scanning

15 μg of each biotinylated cRNA were fragmented at 94° C. for 35 minutes in a buffer solution containing 40 mM Tris-Acetate (pH 8.1), 100 mM KOAc and 30 mM MgOAc. The fragmented cRNA was mixed with hybridization buffer (10 mM MES, 1M NaCl, 20 mM EDTA, 0.01% Tween 20) and was heated at 99° C. for 5 minutes and subsequently at 45° C. for 5 minutes, to then be loaded into the Affymetrix array. The first array in which hybridization was carried out was Affymetrix Test 3. This array allows testing the quality of the RNA prior to expression analysis in the Affymetrix® GeneChip® Rat Genome U34 set (RG-U34).

For hybridization, the arrays were incubated in a rotary oven at 45° C. for 16 hours and with a constant rotation of 60 rpm.

Washing and staining of each array was carried out in the Affymetrix® Fluidics Station. A washing and staining program was used which included:

10×2 washing cycles with SSPE-T 6× (0.9 M NaCl, 60 mM NaH₂PO₄, 6 mM EDTA, 0.01% Tween 20) at 25° C.,

4×15 cycles with 0.1 mM MES, 0.1M NaCl, 0.01% Tween 20 at 50° C., staining of biotinylated cRNA with a streptavidin-phycoerythrin conjugate (10 μg/ml Molecular Probes)

10×4 washing cycles with SSPE-T at 25° C.

staining with an anti-streptavidin antibody (3 μg/ml, Vector laboratories) for 10 minutes

staining with a streptavidin-phycoerythrin conjugate (10 μg/ml, Molecular Probes) for 10 minutes

15×4 washing cycles with SSPE-T at 30° C.

The arrays were scanned at 560 nm using a confocal microscope using laser emission (Agilent GeneArray Scanner). Analysis of the intensity readings was carried out with Microarray Suite 5.0 software. To compare the arrays, the latter were scaled to a total intensity of 100.

### 1.2. Results.

Differential analysis of the dusp6 gene expression in the oligodendrocytes stimulated with regard to the control was carried out from the array comparison data obtained using the Affymetrix software. The parameters taken into account (in the order in which they appear in the list) were: i) Detection. This indicates if the transcribed element is Present (P), Absent (A) or Marginal (M), ii) Change: This indicates if the expression of a certain transcribed element Increases (I), Decreases (D), undergoes No Change (NC), Marginally Increases (MI), or Marginally Decreases (MD), iii) Signal Log Ratio (SLR): This indicates the level of expression change between the baseline (control) and a test sample. This change is expressed as log₂ of the ratio (fold change or number of times the expression of the gene is increased or inhibited in the treated test sample in comparison to the untreated control sample). An SLR value of 1 (equivalent to a fold change of 2) is considered significant for transcribed elements the expression of which increases in comparison to the control, and an SLR value of −1 is considered significant for transcribed elements the expression of which decreases in comparison to the control.

TABLE 2
Results obtained with the Rat Genome U34 array for
dusp6, Genebank accession number U42627.

| | | | SLR | | |
|---|---|---|---|---|---|
| | Signal | Detection | (vs control) | Ratio (=2^{SLR}) | Change |
| Control | 34.1 | P | | | |
| AMPA 2 | 75.3 | P | 0.8 | 1.7 | I |
| AMPA 10 | 101.7 | P | 1.2 | 2.3 | I |
| AMPA 15 | 149.7 | P | 2.1 | 4.3 | I |
| Kainate 2 | 48.4 | P | 0.6 | 1.5 | I |
| Kainate 10 | 43.5 | P | 0.6 | 1.5 | I |
| Kainate 15 | 63.7 | P | 0.9 | 1.9 | I |

### 1.3. Discussion

Differential analysis of the dusp6 gene expression in oligodendrocyte cultures treated with en AMPA with regard to the control showed that dusp6 gene expression levels were increased more than 4 fold (SLR=2.1) in the 15-minute treatment and more than 2 fold (SLR=1.2) in the 10-minute treatment, whereas the expression increase was less than two fold (SLR<1) in treatments with kainate.

### Example 2

### Differential Analysis of DUSP6 Protein Expression in Oligodendrocyte Cultures, Using the Real Time Quantitative RT-PCR Technique

### 2.1. Materials and Methods.

The method used consists of reverse transcription of mRNA to cDNA and its subsequent amplification into a LightCycler equipment (Roche), using SYBR Green for the detection of the amplified product. Real time quantification is carried out, and it allows calculating the relative expression of the sequence in different samples in the linear amplification phase of the reaction.

Samples: Primary oligodendrocyte culture samples treated with AMPA were analyzed. The samples came from cultures different from those analyzed with DNA microarrays. 4 cultures were carried out:

control: untreated cultured oligodendrocytes

ampa 2: oligodendrocytes treated for 2 minutes with 10 μM AMPA and collected after treatment

ampa 10: oligodendrocytes treated for 10 minutes with 10 μM AMPA collected after treatment

ampa 15: oligodendrocytes treated for 15 minutes with 10 μM AMPA and collected 60 minutes later.

kainate 2: oligodendrocytes treated for 2 minutes with 3 μM kainate and collected after treatment

kainate 10: oligodendrocytes treated for 10 minutes with 3 μM kainate and collected after treatment

kainate 15: oligodendrocytes treated for 15 minutes with 3 μM kainate and collected 60 minutes later.

The cells were collected in TRIzol® Reagent (Life Technologies) and were stored at −80° C. until the moment of total RNA extraction.

Real time quantitative RT-PCR. The total RNA of each one of the cultures was obtained by homogenizing the cells in TRIzol® Reagent (Life Technologies) following the supplier's recommendations. The resulting total RNA was cleaned with the RNeasy kit (QIAGEN) (Chomczynski P. et al., Anal. Biochem., 1987, 162: 156; Chomczynski P., Biotechniques, 1993, 15: 532). The total RNA samples were conserved at −80° C. from the moment they were obtained until they were used. The RNA was spectrophotometrically quantified and 5 μg of total RNA were digested with DNase (2 U/μl) of Ambion at a concentration of 2 U per μg of RNA, for 30 minutes at 37° C.

cDNA Synthesis

1 μg of RNA treated with DNAse as a starting material was used for the synthesis of the first cDNA strand with the reverse transcriptase enzyme SuperScript™ II RNase (Life Technologies), using an oligo-dT oligonucleotide as a primer which contained the sequence of the promoter of the phage T7 RNA polymerase. Aliquots of cDNA diluted to the working concentration were used.

Amplification

Synthesized cDNA was amplified using specific primers of the rat dusp6 gene (SEQ ID NO: 1 and SEQ ID NO: 2), and of the gene encoding the rat protein glyceraldehyde 3-phosphate dehydrogenase, Genebank accession number NM_{—}017008 (SEQ ID NO:21 and SEQ ID NO:22). Real time PCR reactions were prepared using the LightCycler-FastStart DNA master SYBR Green I kit (Roche) following the manufacturer's instructions. The amplification program consisted of 195° C. cycle for 10 min (“hot start”) followed by 45 95° C. cycles (denaturation) for 10 seconds, 60° C. (annealing) for 5 seconds, 72° C. (amplification and fluorescence acquisition) for 10 seconds. The melting curve analysis program consisted of a one-pulse cycle of 95° C., 65° C. for 15 seconds, and a 95° C. pulse during the amplification and acquisition step.

Quantification

First, specificity of the PCR products was determined by analyzing the melting curves. Subsequently, as a relative measurement of gene expression, the ratio between the abundance of dusp6 transcribed mRNAs and the abundance of GAPDH transcribed mRNAS was calculated, and the data of the ratio was standardized in each one of the treated samples on the basis of control sample values. To calculate efficiency of the PCR reactions (dusp6 and GAPDH), a standard curve was constructed for each gene sequence carried out with cDNA serial dilutions. The template cDNA concentrations for the reactions on the standard curve were arbitrarily given the values 2×, 0.5×, 0.2× and 0.02×. The crossing point values for each one of the reactions were used to construct the standard curve: y=m×+b, where m is the slope and b is the intersection point of the line on the axis of ordinates. Starting RNA concentration values in each sample were obtained from the data of the line using the following formula:

[RNA]=10^{(Cp-b)/m}

where Cp is the crossing point.

The units of the calculated concentration are the same as those used for constructing the standard curve, therefore in this case they are arbitrary units disappearing in subsequent calculations as they are ratios.

The concentration values obtained for dusp6 in each sample were standardized in comparison to the values obtained for GAPDH (used in this sense as a “housekeeping” gene, the expression of which does not vary as a consequence of treatment).

The change rate at each point was obtained by dividing the concentration values standardized in comparison to the concentration values in the control.

### 2.2. Results.

The real time quantitative RT-PCR results were used to obtain the dusp6 gene expression change rate in treated oligodendrocyte samples with regard to the untreated control.

Melting Curve Analysis

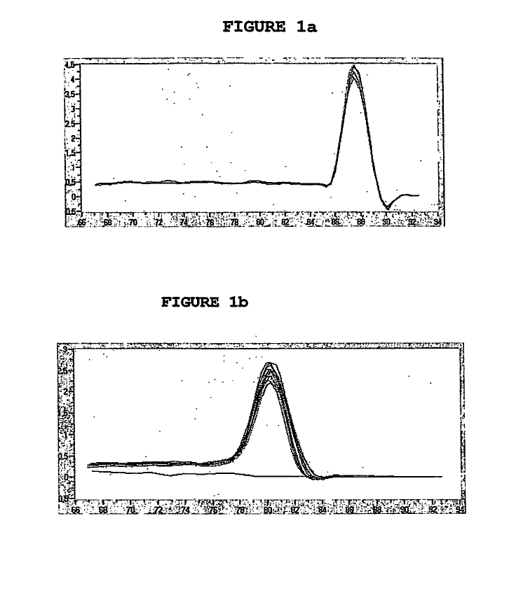

PCR product analysis showed the specific amplification of a product with a melting temperature similar to that estimated according to the software used for primer design, PrimerExpress (Applied Biosystems) (FIG. 1).

Calculation of Efficiency of PCR Reactions

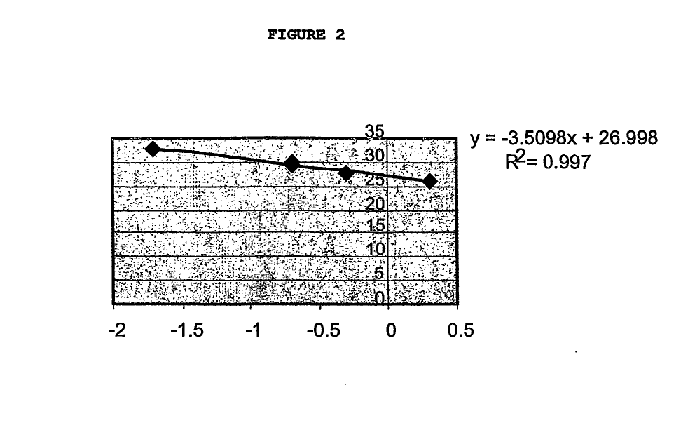

Two replicas of each one of the 4 cDNA dilutions were amplified, and the cutting points (Cp) of each one of them were represented in a graph with regard to the logarithm of the cDNA concentration to construct the standard line (Table 3 and FIG. 2).

TABLE 3
Crossing points (Cp) of the amplification of the 4 cDNA dilutions
for each replica amplified with the dusp6 gene primers.

| [RNA] | Log [RNA] | Cp |
|---|---|---|
| 2x | 0.30103 | 25.91 |
| 2x | 0.30103 | 26.04 |
| 0.5x | −0.30103 | 27.88 |
| 0.5x | −0.30103 | 27.88 |
| 0.2x | −0.69897 | 29.54 |
| 0.2x | −0.69897 | 29.72 |
| 0.02x | −1.69897 | 32.90 |
| 0.02x | −1.69897 | 32.95 |

Quantification of the dusp6 Gene Expression Change

Two replicas of each sample (control, AMPA2, AMPA10 and AMPA15) were amplified with the dusp6 and GAPDH specific primers. Expression changes of the dusp6 gene in the treated samples with regard to the untreated control (table 5) were calculated from the cutting points generated in these amplifications (table 4).

TABLE 4
Experimental dusp6 crossing points in the 4 samples

| Replicas | Control | AMPA2 | AMPA10 | AMPA15 |
|---|---|---|---|---|
| 1 | 31.94 | 30.46 | 32.15 | 29.73 |
| 2 | 31.65 | 30.54 | 32.31 | 29.61 |
| Mean | 31.795 | 30.5 | 32.23 | 29.67 |
| SD | 0.205 | 0.056 | 0.113 | 0.084 |

TABLE 5
Dusp6 overexpression values (Fold Change) in the 3
treated samples with regard to the untreated control

| Sample | Ratio |
|---|---|
| AMPA 2 | 1.7 |
| AMPA 10 | 0.9 |
| AMPA 15 | 2.7 |

### 2.3. Discussion.

The results confirmed the data obtained in differential gene expression analysis with DNA-chips (example 1), thus, dusp6 expression was increased 2.7 fold in treatment with AMPA15. The different degree of sensitivity of the two techniques could explain the lack of concordance between results of the quantitative RT-PCR and of the DNA-chip for the AMPA10 sample in which a slight overexpression was observed in example 1.

### Example 3

### Cell Death Inhibition in Oligodendrocytes Treated with AMPA after Incubation with Antisense Oligonucleotides Against the dusp6 Gene

### 3.1. Materials and Methods.

To evaluate if dusp6 expression blocking inhibits cell death of oligodendrocytes treated with AMPA, antisense oligonucleotides (ODNs) capable of blocking said gene expression were used. The working model used in this assay is a model based on the cell death of oligodendrocytes, a typical model for study the neurodegenerative phase of said demyelinating diseases.

Oligonucleotide design was carried out based on the secondary structure of the RNA, trying to prevent sequences presenting a high internal hybridization percentage, and with the aid of AO predict (http://www.cgb.ki.se/AOpredict/) computer software. To improve efficacy and capability to degrade RNA and to reduce side effects, last-generation oligonucleotides were designed, presenting two types of modifications; 2′-O-Methyl groups in the 6 first and last bases, and phosphorothiodated groups in the 8 intermediate bases. Furthermore, the oligonucleotides were labeled with Texas Red to be able to identify the cells transfected with said oligonucleotides.

Three antisense ODNs, for the purpose of identifying active sequences, plus a sense ODN, used as a negative control, were designed. To improve penetration thereof, the ODNs were transfected using the Lipofectin kit (Invitrogen).

Table with the antisense oligonucleotide sequences

| | |
|---|---|
| ODN1- | SEQ ID NO: 23 |
| ODN2- | SEQ ID NO: 3 |
| ODN3- | SEQ ID NO: 4 |

24 hours after seeding, the oligodendrocytes were treated with a mixture of ODs at different concentrations coupled to the transfection kit liposomes.

After 24 hours, the cells were rinsed and were left another 24 hours before the experiment to ensure total blocking of the protein under study. Subsequently, the excitotoxic stimulus, AMPA 10 μM+CTZ 100 μM, was added during 15 minutes, and the protecting effect of the different ODNs after 6 hours was assessed.

### 3.2. Results.

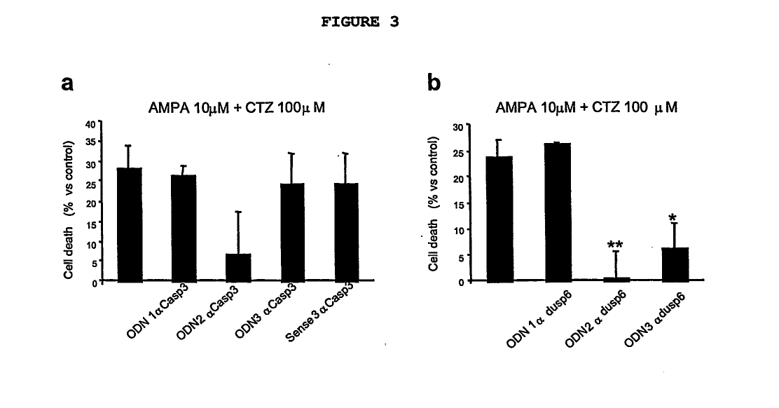

ODN transfection percentages of about 50% were observed. To complete the technique, antisense ODNs were designed against caspase-3, mediator of oligodendroglial death process induced by AMPA 10 μM+CTZ 100 μM. From among the 3 ODNs designed against caspase-3, ODN 2 achieved blocking the effect of the excitotoxic stimulus, showing validity of the method (FIG. 3A).

Then, antisense ODNs were designed against dusp6, and oligodendrocyte cultures were transfected according to that previously described. Thus, oligodendroglial death after stimulation with AMPA 10 μM+CTZ 100 μM was significantly inhibited in the presence of the antisense ODNs 2 (p<0.01; n=5) and 3 (p<0.05; n=3) against dusp6 (FIG. 3B).

### 3.3. Discussion.

The assays carried out showed that dusp6 contributes to the oligodendroglial death process, since blocking of the expression of said gene inhibited cell death induced by treatment with AMPA.

The results also suggested that the dusp6 gene specific antisense oligonucleotide, which inhibited cell death in oligodendrocytes treated with AMPA after incubation, could be a pharmacologically active molecule in treatment of demyelinating diseases, preferably multiple sclerosis.

### Example 4

### Oligodendrocyte Death Triggered by AMPA is Potentiated by Inhibition of Mitogen-Activated Protein Kinase (MEK)

### 4.1. Materials and Methods.

In the previous example (Example 3) we have demonstrated that blockade of DUSP6 synthesis by oligodeoxynucleotides prevents oligodendrocyte death by excitotoxycity. DUSP6 is a negative regulator of the MAPK/ERK signaling pathway which promotes cell survival, as documented above in the Background of the Invention section. Because of that, we tested if the pharmacological inhibition of this pathway potentiates oligodendrocyte death triggered by activation of AMPA receptors.

To block the MAPK/ERK pathway, oligodendrocytes were incubated with UO126, a dual MEK1 & 2 inhibitor, for 15 min prior to the excitotoxic stimulus. Subsequently, cells were exposed to AMPA 10 μM+CTZ 100 μM during 15 minutes in the presence or absence of UO126, and cell viability was assessed 24 h later by the MTT method.

### 4.2. Results.

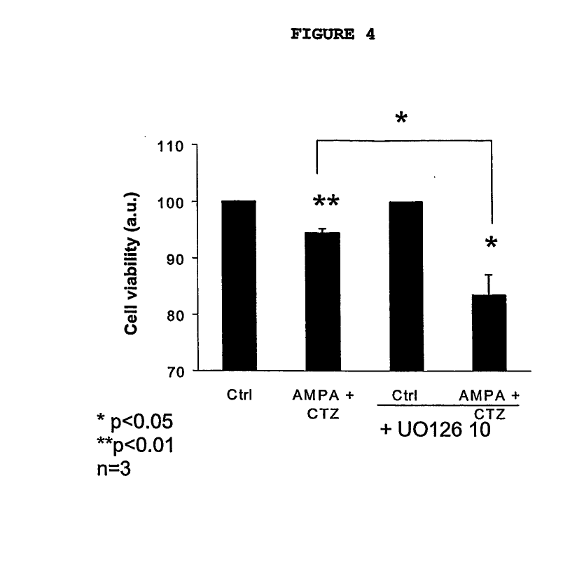

The MEK inhibitor UO126 (10 μM; FIG. 4) did not affect oligodendroglial viability under control conditions. However, oligodendroglial cell death induced by AMPA 10 μM+CTZ 100 μM was increased three-fold in the presence of UO126 (p<0.05; n=3 in triplicate; FIG. 4A).

### 4.3. Discussion.

The assays carried out showed that inhibition of the MAPK/ERK kinase pathway potentiates oligodendrocyte death induced by AMPA receptor stimulation. This indicates that this pathway is activated as a compensatory mechanism to promote cell survival upon excitotoxic insults. In addition, these results provide further evidence that DUSP6 increased expression has a deleterious effect on cell viability by modulating negatively the MEK pathway. Moreover, these findings are also in accordance with data illustrated in Example 3, which shows that dusp6 gene silencing is protective in oligodendrocytes. Taken together, the evidence provided here indicates that excitotoxicity in these cells increases the expression of DUSP6/MKP-3, and as a consequence, reduces the activity of the MAPK/ERK pathway. Further inhibition of this pathways with UO126 aggravates the outcome of excitotoxicity in oligodendrocytes.

### Example 5

### Comparison Analysis of Rat and Human dusp6 Sequences

### 5.1. Materials and Methods

For the purpose of comparing the degree of homology of the dusp6 gene human and rat sequences, a nucleotide sequence alignment was carried out. The rat dusp6 sequence was compared to the human dusp6 sequence. The nucleotide sequences as well as the amino acid sequences were compared.
The nucleotide sequences to compare were:

Rat dusp6, GenBank accession number NM_{—}053883;

Human dusp6, GeneBank accession number NM_{—}001946.

The amino acid sequences to compare were:

Rat DUSP6, GenBank accession number NP_{—}446335;

Human DUSP6, GeneBank accession number NP_{—}001937.

Alignment was carried out using computer tools available on web page http://biobug.life.nthu.edu.tw/˜tswang/pw-fasta/.

### 5.2. Results

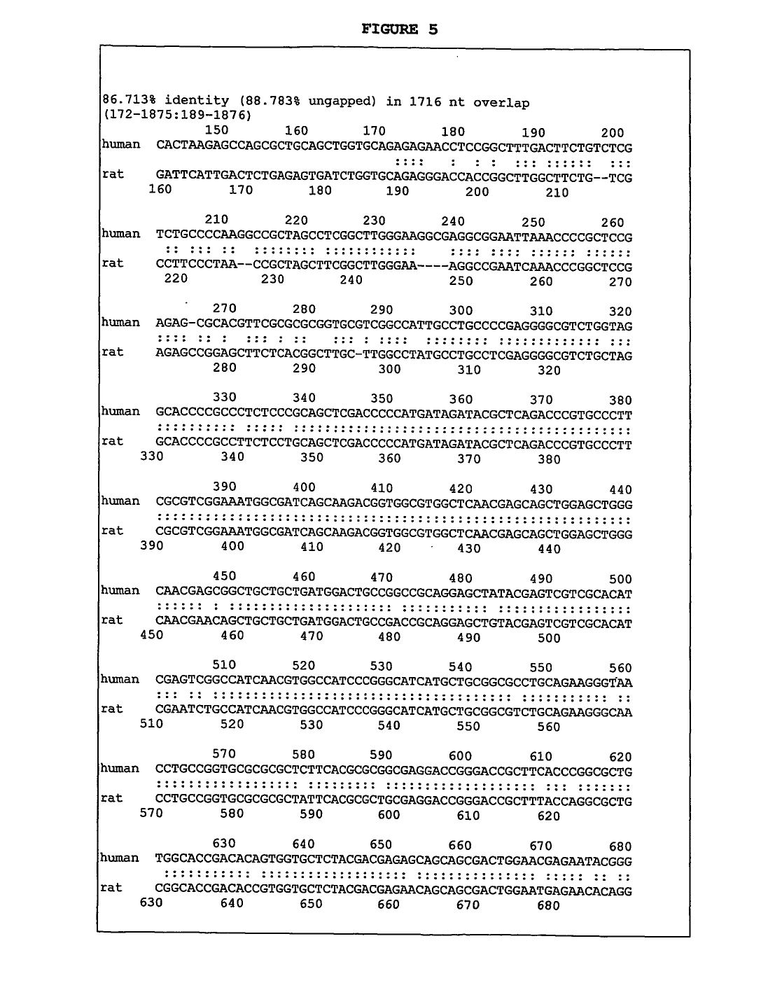

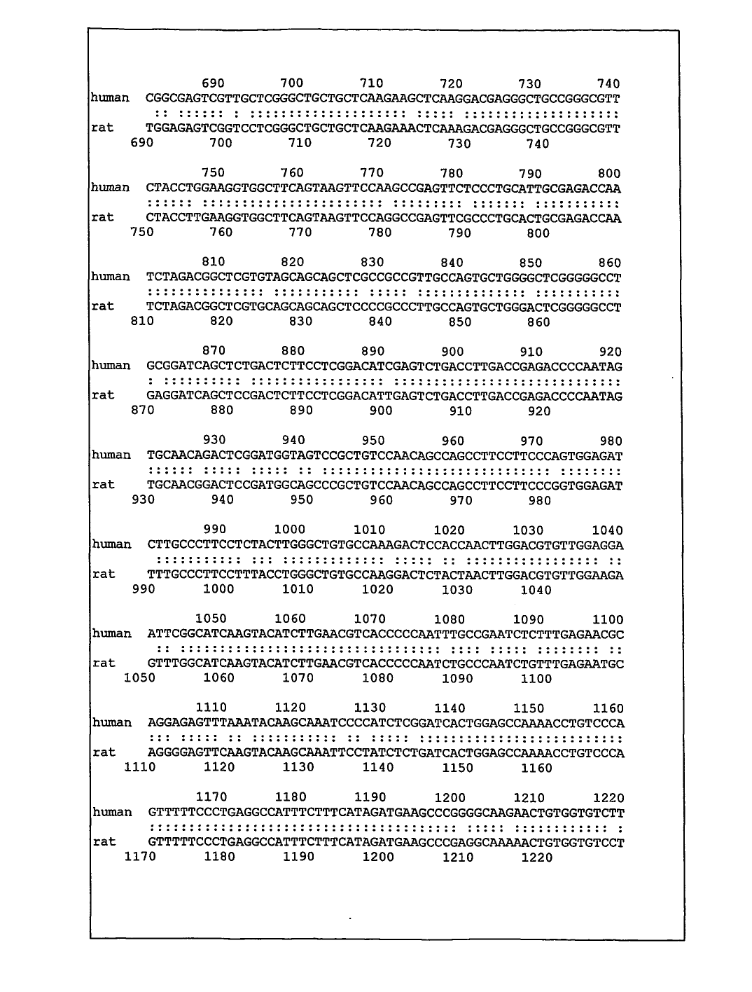

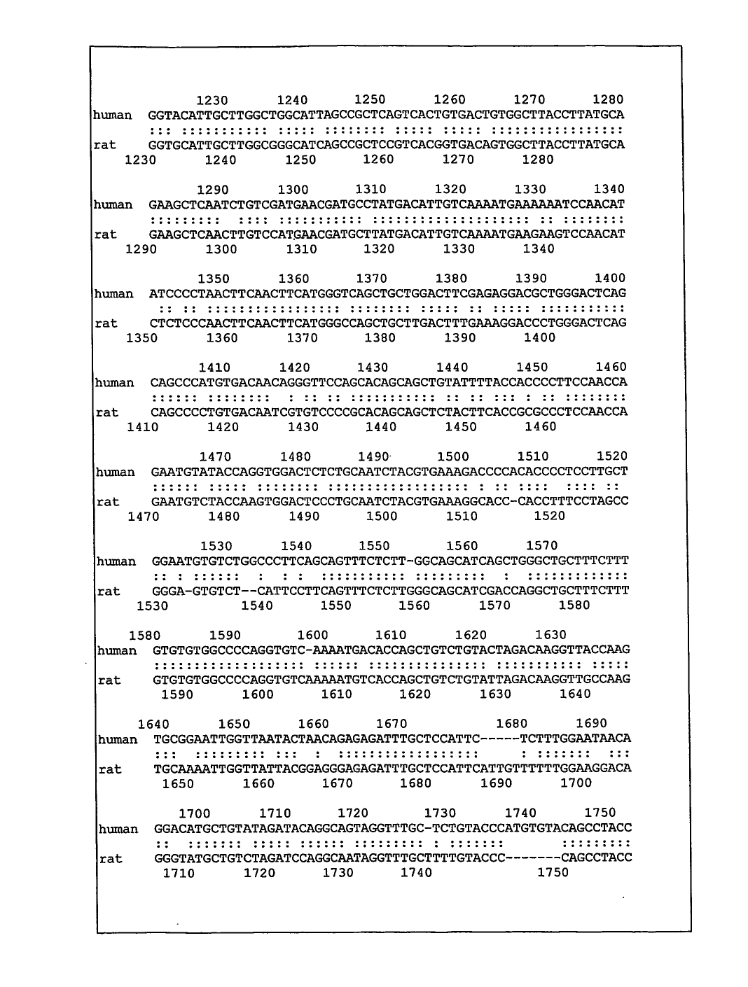

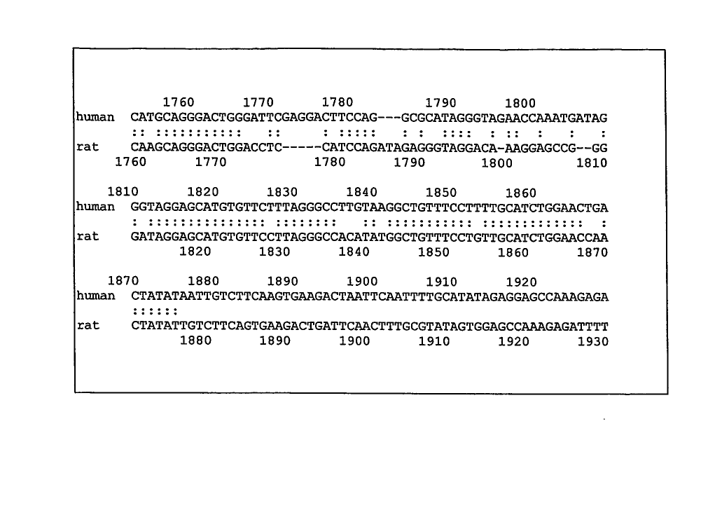

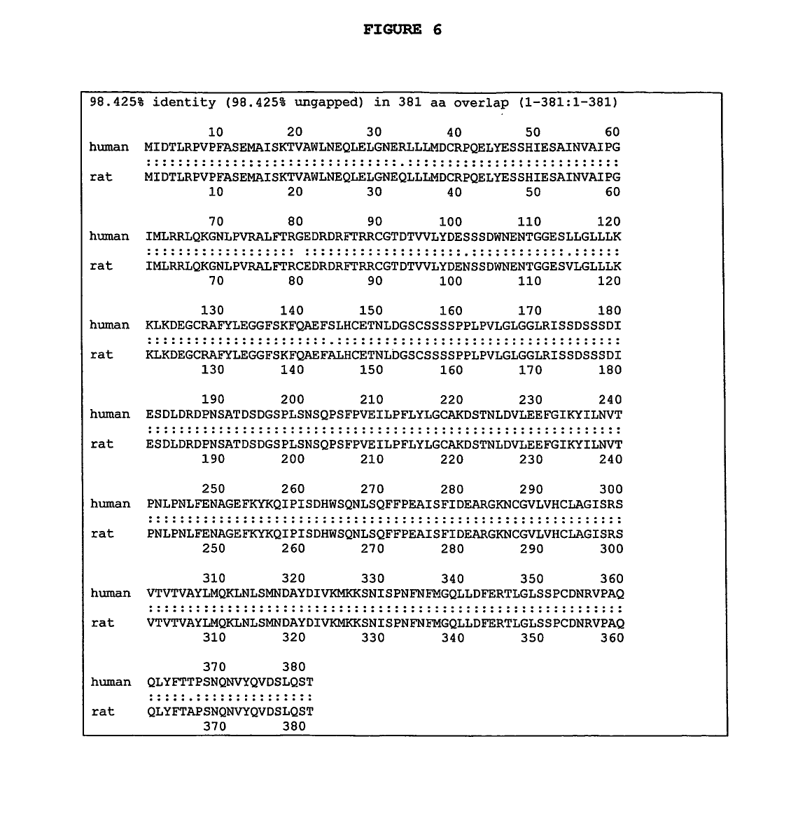

Results of the nucleotide and amino acid alignments are shown in FIGS. 5 and 6, respectively.

### 5.3. Discussion

Alignment of the NM_{—}001946 (human dusp6) and NM_{—}053883 (rat dusp6) sequences revealed an 86.713% degree of identity on a sequence overlap of 1716 nucleotides.

With regard to the amino acid sequence comparison, the degree of identity between the NP_{—}001937 (human DUSP6) and NP_{—}446335 (rat DUSP6) sequences was 98.425% on an overlap of 381 amino acids.

### Example 6

### Dusp6 Gene Expression in Postmortem Human Tissue from Controls and Multiple Sclerosis Subjects

### 6.1. Materials and Methods.

Quantitative real-time RT-PCR studies were performed to evaluate if dusp6 expression was altered in the central nervous system of multiple sclerosis patients, as compared to control individuals matched by age and gender, who had died with no neurological disease symptoms.

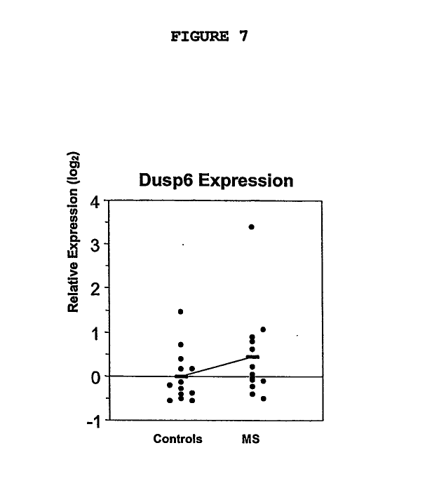

Tissue samples originated from optic nerves were dissected out within 8 hours postmortem and snap frozen in liquid nitrogen. Total RNA was extracted with Trizol (Invitrogen, Paisley, UK) and cDNA was synthesized by reverse transcription (Applied Biosystems, Madrid, Spain) using random hexamers. Real-time quantitative RT-PCR was carried out in an ABI PRISM 7000 Sequence Detection System instrument (Applied Biosystems) using the primers SEQ ID NO:24 and SEQ ID NO:25 designed using the PrimerExpress software (Applied Biosystems). The amount of cDNA was calculated from the appropriate standard curve of a stock cDNA obtained from human cerebral cortex. Cyclophilin C, GAPDH, r18S, ubiquitin C, HRPT1 and 2-β-microglobulin housekeeping genes were used as endogenous references to normalize the variability in the initial quantities of total RNA, so that accurate comparison of gene expression levels could be made among the different samples. Target genes were normalized by means of a normalization factor, based on the geometric mean of multiple internal control genes (Vandesompele et al., 2002 Genome Biol 3:1-12.) obtained from the five internal control genes. The results are shown in FIG. 7; data are expressed as fold change in gene expression compared to the matched-controls. All results are expressed as mean ±SEM and statistical comparisons made by non paired, one tail Student's t test.

### 6.2. Results.

Quantitative RT-PCR analysis revealed that dusp 6 gene in all the samples analysed is expressed in variable amounts as compared to the five housekeeping genes used. In average, Multiple sclerosis patients samples had an increased expression of 43.83±0.28% over controls (p=0.074, n=13; FIG. 7).

### 6.3. Discussion.

The above described assays showed that the expression of dusp6 is increased in samples of optic nerve from multiple sclerosis patients. Because oligodendrocytes are the major cellular component of the optic nerve, the increased expression of dusp6 in the nerve most probably corresponds to higher transcript levels of this gene in these cells. This suggests that oligodendrocytes in the multiple sclerosis patients samples are at a higher risk of undergoing cell death, since dusp6 gene product is associated to cellular damage (Rossig et al., 2000, J. Biol. Chem. 275:25502-25507).

1. An in vitro method for detecting the presence of a demyelinating disease in an individual, for determining the stage or severity of said disease in the individual, or for monitoring the effect of the therapy administered to an individual presenting said disease, comprising:
a) detecting and/or quantifying of the DUSP6 protein, the dusp6 gene mRNA, or the corresponding cDNA in a sample of said individual, and
b) comparing the DUSP6 protein amount, of the dusp6 gene mRNA amount, or of the corresponding cDNA amount detected in a sample of an individual, with the DUSP6 protein amount, with the dusp6 gene mRNA amount, or with the corresponding cDNA amount detected, respectively, in samples from control individuals or with normal reference values.

2. A method according to claim 1, wherein the demyelinating is selected from the group consisting of multiple sclerosis, Devic's syndrome, Baló disease, Marchiafava-Bignami disease, central pontine myelinolysis, acute disseminated encephalomyelitis, and acute necrotizing hemorrhagic encephalomyelitis.

3. A method according to claim 1, wherein said sample is selected from the group consisting of serum, urine, saliva, feces, and cerebrospinal fluid.

4. A method according to claim 3, wherein said sample is obtained by surgical resection.

5. A method according to claim 1, wherein said sample is obtained from an individual who has not previously been diagnosed with a demyelinating disease.

6. A method according to claim 1, wherein said sample to analyze is obtained from an individual who has previously been diagnosed with a demyelinating disease.

7. A method according to claim 1, wherein said sample to analyze is obtained from an individual undergoing treatment, or who has been previously treated against a demyelinating disease.

8. A method according to claim 1, further comprising carrying out an extraction of the sample to obtain a protein extract to obtain an extract consisting of total RNA.

9. A method according to claim 8, characterized in that the detecting of the DUSP6 protein comprises contacting the protein extract of the sample with a composition of one or more specific antibodies against one or more epitopes of the DUSP6 protein, and quantifying the complexes formed by the antibodies and DUSP6 protein.

10. A method according to claim 9, characterized in that said antibodies comprise a species selected from the group consisting of monoclonal antibodies, polyclonal antibodies, intact fragments thereof, recombinant fragments thereof, “combibodies,” Fab antibody fragments, and scFv antibody fragments.

11. A method according to claim 10, characterized in that the complexes formed by the antibodies and the DUSP6 protein are quantified using a technique selected from the group consisting of Western-blot, ELISA (Enzyme-Linked Immunosorbent Assay), RIA (Radioimmunoassay), Competitive EIA (Competitive Enzyme Immunoassay), DAS-ELISA (Double Antibody Sandwich-ELISA), immunocytochemical techniques, immunohistochemical techniques, techniques based on the use of protein biochips, techniques based on the use of microarrays including specific antibodies, assays based on precipitation with colloidal gold, affinity chromatography techniques, ligand binding assays and lectin binding assays.

12. A method according to claim 8, characterized in that the detecting of mRNA comprises a first amplification step of the mRNA included in the total RNA extract, and a second quantification step of the amplification product of the mRNA of the dusp6 gene.

13. A method according to claim 12, characterized in that the amplification is carried out by means of RT-PCR using oligonucleotide primers wherein the sequences of the primers used to amplify the dusp6 gene sequence are selected from the group consisting of SEQ ID NO:1, SEQ ID NO:2, and any other primer pair amplifying dusp6.

14. A method according to claim 8, characterized in that the detecting of mRNA is carried out by specific probes of mRNA or the corresponding cDNA probes of the dusp6 gene.

15. A method according to claim 8, characterized in that mRNA detection is carried out by means of real time quantitative RT-PCR (Q-PCR).

16. (canceled)

17. An in vitro method for identifying and evaluating the efficacy of an agent for therapy of demyelinating diseases, said method comprising:
a) treating a primary culture of rat optic nerve oligodendrocytes with stimuli relevant to demyelinating diseases to produce a culture of stimulated oligodendrocytes,
b) detecting and quantifying changes in the dusp6 gene or DUSP6 protein expression in cells of the culture of stimulated oligodendrocytes in response to said stimuli,
c) contacting the culture of stimulated oligodendrocytes with the agent under conditions and for time suitable for permitting interaction between the stimulated oligodendrocytes and the agent to form interacting stimulated oligodendrocytes,
d) detecting and quantifying the dusp6 gene or DUSP6 protein expression levels in the culture of interacting stimulated oligodendrocytes, and
e) comparing the expression levels obtained in step d) with the corresponding levels in the culture of stimulated oligodendrocytes not treated with the agent.

18. (canceled)

19. A method for the treatment of the neurodegenerative phase of demyelinating diseases, which comprises the administration of an agent that inhibits DUSP6 protein expression and/or activity, or that inhibits the lethal effects of induction of DUSP6 protein expression.

20. The method according to claim 19 wherein said agent is selected from the group consisting of:
a) an antibody, or combination of antibodies, specific against one or more epitopes present in the DUSP6 protein, wherein said antibody comprises a human monoclonal antibody, a humanized monoclonal antibody a fragment of the antibody, a single-chain antibody or an anti-idiotype antibody,
b) cytotoxic agents selected from the group consisting of toxins, molecules with radioactive atoms, and chemotherapeutic agents, and
c) DUSP6 protein antagonist compounds inhibiting one or more of the DUSP6 protein functions.

21. The method according to claim 19, which comprises the additional administration of a DUSP6 protein function inhibitor.

22. A dusp6 specific antisense oligonucleotide selected from the group consisting of SEQ ID NO:3 and SEQ ID NO:4.

23. A kit for an in vitro method of detecting the presence of a demyelinating disease in an individual, said kit comprising an antibody that specifically recognizes the DUSP6 protein and a carrier in suitable packing.

24. A kit for an in vitro method of detecting the presence of a demyelinating disease in an individual, said kit comprising a primer pair designed to specifically amplify a nucleic acid having a sequence that is specific to the dusp6 gene.

25. A kit according to claim 24, wherein the sequence of the primer pair is selected from the group consisting of SEQ ID NO: 1 and SEQ ID NO: 2.

26. A kit according to claim 23 that is employed to detect the presence of demyelinating diseases in an individual, to determine the stage or severity of said conditions in an individual or to monitor the effect of the therapy administered to the individual with said conditions.

27. A method according to claim 8, characterized in that the detecting of corresponding cDNA dusp6 gene comprises a first amplification step of the corresponding cDNA synthesized by reverse transcription of the mRNA, and a second quantification step of the amplification product of the cDNA of the dusp6 gene.
